(19) United States
(12) Patent Application Publication
(10) Pub. No.: US 2020/0268637 A1
(43) Pub. Date: Aug. 27, 2020

(54) Ultrafine Polyhydroxyalkanoates

(71) Applicant: Micro Powders, Inc., Tarrytown, NY (US)

(72) Inventor: Richard John Czarnecki, Tarrytown, NY (US)

(21) Appl. No.: 16/545,113

(22) Filed: Aug. 20, 2019

Related U.S. Application Data
(60) Provisional application No. 62/810,636, filed on Feb. 26, 2019.

(51) Int. Cl. A61K 8/85; A61K 8/02; A61Q 1/12; A61Q 19/00

(52) U.S. Cl. CPC A61K 8/85; A61Q 19/007; A61Q 1/12; A61K 8/0225

(57) ABSTRACT

A powder for use in cosmetic and personal care products containing ultrafine polyhydroxyalkanoate (PHA), ultrafine polyhydroxybutyrate (PHB), or ultrafine polyhydroxybutyrate-co-hydroxyvalerate (PHBV).

### CROSS-REFERENCE TO RELATED APPLICATIONS

This patent application claims the benefit, under 35 U.S.C. § 119(e), of U.S. Provisional patent application Ser. No. 62/810,636, filed on Feb. 26, 2019, the content of which is incorporated herein by reference in its entirety.

### TECHNICAL FIELD

The present invention relates to certain novel polymer powder compositions suitable for personal and cosmetics compositions. The present invention also relates to cosmetic compositions comprising such novel polymer compositions. The present invention further relates to processes for micronizing polymer pellets, methods of making cosmetic and personal care compositions and the methods of using such cosmetic compositions in topical applications.

More particularly, the present invention relates to cosmetic compositions, including, but no limited to, fine particle cosmetic products, loose and packed cosmetic powders, foundations, eye shadows, creams, liquids, gels, lotions and compositions suitable for keratin materials made from ultrafine biodegradable polymers. The compositions according to the inventions are intended more particularly for the keratin materials of human beings, such as the skin (including the scalp), the nails and keratin fibers, and/or mucous membranes.

### BACKGROUND

### Prior Art Cosmetics

Over the years, cosmetics have been developed in many different formulations and in many different forms, including, but not limited to, a powder, liquid, gel or cream. These different formulations and forms have been used for a host of topical uses.

Cosmetic powders have been long known and commercially available in both loose and pressed powder form. Cosmetic powders have been used for a variety of topical application such as to the face, lips, around the eyes, nails, scalp and other body locations. The uses of these cosmetic powders include, but are not limited to, reducing, inhibiting or preventing moisture loss from the skin for an extended period of time, absorbing excess sebum/oil from the skin to reduce shine, and filling in blemishes.

As will be seen none of the prior art discloses the present invention.

### Biodegradable Polymers

In recent years, interest in protecting the environment by not only using products made from natural renewable resources, but also products that decompose into environmentally friendly constituents has been steadily and rapidly increasing. Green movements, initiatives and regulations have sprung up in almost every developed country. Consumers, in particular, have also expressed their desire for products that are environmentally friendly while providing the same results that products made from synthetic materials. This desire by consumers for environmentally friendly products has spread to cosmetics and health care products.

Over the years, biodegradable polymers such as polylactic acid (PLA) have been employed in several products. Because polylactic acid (PLA) has high transparency, toughness, and is easily hydrolyzed in the presence of water, blends of stereocomplexes of polylactic (PLA) have been woven into shirts, microwavable trays, hit-fill applications and even engineering plastics.

Because polylactic acid (PLA) is 100% compostable it can also be employed in the preparation of bioplastics, useful for producing loose-fill packaging, compost bags, food packaging, and disposable tableware.

In addition, because polylactic acid (PLA) easily decomposes and is absorbed in a living body without exerting toxicity it has been used in vivo. For example, polylactic acid (PLA) is currently used in a number of biomedical applications, such as sutures, stents, dialysis media, tissue scaffolds and drug delivery devices.

More recently, attention has been paid to the broad issue of plastic waste in the ecosystem. In particular, environmental groups identified personal care products containing plastic microbeads as a source of plastic pollution in the world's waterways. These particulates, usually based on polyethylene, had been used for many years in rinse-off cosmetic and personal care products as a scrubbing and exfoliating agent.

In December 2015, the United States House and Senate passed, unanimous consent, H.R. 1321, the Microbead-Free Waters Act of 2015. The legislation created a pragmatic phase-out process for solid plastic microbeads in personal care cleansing products and preempts state and local restrictions on microbeads. The bill bans the use of solid plastic particles in rinse off products, and fully implements as of Jul. 1, 2019.

The bill defines a microbead as follows: “The term ‘plastic microbead’ means any solid plastic particle that is less than five millimeters in size and is intended to be used to exfoliate or cleanse the human body or any part thereof”

Since polylactic acid (PLA) is polymerized from repeat units of a monomer (namely lactic acid/lactide), this substance could be considered a plastic material and therefore may be in conflict with the 2015 Act. Additionally, and importantly, polylactic acid (PLA) does not biodegrade in freshwater or marine environments.

Accordingly, the use of exfoliating agents based on plastic microbeads has dramatically declined around the world. Instead, manufacturers are trying to find suitable exfoliating agents based on waxes, biodegradable materials, and natural materials.

While polyethylene is now sparsely used in a coarser form as an exfoliating powder, it is still widely used in an ultrafine form in non-rinse-off cosmetics and personal care products. Polyethylene provides dry binding, creamy aesthetics, and other desirable end properties to cosmetics that included pressed powders, lotions, lipsticks, and hair care products. Non-rinse-off products are not regulated with regards to these issues, but industry and consumers increasingly prefer to seek alternatives to plastic powders, be they ultrafine or coarse, for use in cosmetics and personal care products.

Ultrafine polytetrafluorethylene (PTFE) has also been widely used in an ultrafine form in non-rinse-off cosmetics and personal care products. PTFE provides excellent tactile properties, skin feel, and lubricity in cosmetic products that include pressed powders, lotions, gels, and other products. PTFE is a synthetic plastic and is losing favor with customers who prefer products based on natural materials. More importantly, PTFE powders can contain potentially dangerous side-products that include polyfluorooctanoic acid (PFOA) and longer carbon chain analogs.

In summary, customers are looking for natural alternatives to synthetic ultrafine powders that present potential risks to both the environment and human health. They want a material with similar tactile and aesthetic properties without the risks.

Polyhydroxyalkanoates or PHAs are polyesters produced in nature by numerous microorganisms, including through bacterial fermentation of sugar. When produced by bacteria they serve as both a source of energy and as a carbon store. Many different monomers can be combined within this PHA family to give materials with extremely different properties. Within this PHA family are the substances poly(3-hydroxybutyrate) (PHB) and poly(3-hydroxybutyrate -co-3-hydroxyvalerate) (PHBV). PHAs are unique in that they can be demonstrated to biodegrade in both freshwater (OECD 302) and marine (OECD 306) environments, making them an ideal material as a plastic replacement.

PHAs has been manufactured and sold as a coarse exfoliating powders that are fully biodegradable. However, exfoliating powders are extremely coarse and are not suitable for all personal care products.

However, the prior art does not disclose a suitable fully biodegradable ultrafine powder that provides the benefits of the known plastic ultrafine powders. The present invention solves these issues through the use of ultrafine PHA for topical cosmetic and personal care applications. An ultrafine cosmetic powder ideally has a top particle size which is below 125 μm. Mean particle size can range in particular from about 0.1 to about 44 microns, preferably with a mean particle size of 5 to 20 microns with a maximum particle size of 44 microns, and more preferably with a mean particle size of 8 to 12 microns with a maximum particle size of 31.11 microns.

The prior art does not disclose the use of ultrafine polymers that are substantially biodegradable, that are made from annual renewable resources and that provide efficacious results for use in a skincare product. Likewise, none of the prior art discloses the use of ultrafine PHA, including, PHB and/or PHBV, in cosmetic compositions, and more specifically as an ultrafine powder filler in a cosmetic composition.

### SUMMARY

The needs set forth herein as well as further and other needs and advantages are addressed by the present embodiments, which illustrate solutions and advantages described below.

It is the object of the invention to provide novel methods for deriving ultrafine PHA to make it suitable for topical applications.

The present inventor has also discovered novel PHA powder compositions in ultrafine form suitable for topical use, including, but not limited to, use in skincare products, loose and packed cosmetic powders, lotions, hair care products, and other cosmetic compositions. The tactile properties of these natural PHA powders are surprisingly similar to their synthetic analogs, including polyethylene and PTFE, providing a lubricious, creamy, and slippery aesthetic to personal care products.

It is another object of the invention to provide processes for caring for the keratin materials of human beings, comprising the application of a composition containing the polymer compositions defined above to the keratin materials.

It is yet another object of the present inventions to provide a process for making cosmetic compositions using the ultrafine PHA powder compositions.

The cosmetic powder composition of this invention may be any suitable cosmetic powder composition for application to any suitable area of skin, such as for application to the face, lips, nose, around the eye, the scalp, or any other suitable body area.

The biodegradable powder compositions of the present invention are designed to provide increased slip properties and enhanced texture to the cosmetic and personal care formulations. The ultrafine PHA powder compositions of the present invention are ideally suited as binders in pressed powder formulations.

Further, the cosmetic compositions comprising the ultrafine biodegradable polymers afford good cosmetic properties while being made from annual renewable resources that degrade into benign constituents. The compositions of the present invention give cosmetic compositions that are easy to apply to the skin surface and provide good feel.

More specifically, the cosmetic compositions of the present invention comprise ultrafine PHA particles of various sizes depending on the intended application. These compositions may also comprise other additives that are normally found in cosmetic compositions, again depending on the intended application.

The powder for use in cosmetic and personal care products of one embodiment, according to the present teaching, includes ultrafine polyhydroxyalkanoate. The ultrafine polyhydroxyalkanoate is selected from a compound of monomer unit:

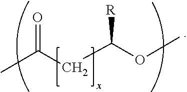

The x is selected from the group consisting of 1, 2, 3, and 4. The R group is selected from the group consisting of hydrogen, alkyl, aryl, alkenyl, aralkyl, haloalkyl, halogen, cyano, radical containing epoxy, ether, acyl, ester, and carboxyl.

In a further embodiment, the x of the ultrafine polyhydroxyalkanoate is selected from the group consisting of 1 and 2, and the R group is selected from the group consisting of hydrogen and alkyl.

In a further embodiment, the ultrafine polyhydroxyalkanoate comprise at least one block constructed from monomers. The monomers are selected from the group consisting of 3-hydroxybutanoic acid, 4-hydroxybutanoic acid, 2-hydroxyvaleric acid, 3-hydroxyvaleric acid, 4-hydroxyvaleric acid, 5-hydroxyvaleric acid, 3-hydroxyhexanoic acid, and 3-hydroxyoctanoic acid.

In a further embodiment, the ultrafine polyhydroxyalkanoate is a polymer selected from the group consisting of poly-3-hydroxybutyrate, poly-3-hydroxybutyrate -co-3-hydroxyvalerate, poly-3-hydroxybutyrate-co-4-hydroxybutyrate, and poly-3-hydroxybutyrate-co-3-hydroxyvalerate-co-4-hydroxybutyrate.

In a further embodiment, the ultrafine polyhydroxyalkanoate has a maximum particle size below 125 microns.

In a further embodiment, the ultrafine polyhydroxyalkanoate has a mean particle size ranging from 0.1 microns to 44 microns.

In a further embodiment, the ultrafine polyhydroxyalkanoate has a mean particle size of 5 microns to 20 microns.

In a further embodiment, the ultrafine polyhydroxyalkanoate is produced by mechanical milling of coarser powders or pellets.

In a further embodiment, the ultrafine polyhydroxyalkanoate is produced by air milling of coarser powders or pellets.

In a further embodiment, the ultrafine polyhydroxyalkanoate is produced by wet high shear milling of coarser powders or pellets.

In a further embodiment, the ultrafine polyhydroxyalkanoate is produced from a mixture of ultrafine and coarse polyhydroxyalkanoate powder by a particle size reduction process.

In a further embodiment, the ultrafine polyhydroxyalkanoate is produced directly from a bio-fermentation process, purified, and dried.

In a further embodiment, the ultrafine polyhydroxyalkanoate is produced by mechanical or air milling to deagglomerate primary particles and further reduce particle size.

In a further embodiment, the ultrafine polyhydroxyalkanoate is produced by wet high shear milling of coarser powders or pellets.

In a further embodiment, the powder comprises a range of 1 to 50 percent by weight of said ultrafine polyhydroxyalkanoate; and a range of 50 to 99 percent by weight of at least one cosmetic powder base components selected from a group consisting of pigments, waxes, solvents, resins, fillers, binders, and mixtures thereof.

The powder for use in cosmetic and personal care products of one embodiment, according to the present teaching, includes ultrafine polyhydroxybutyrate. The ultrafine polyhydroxybutyrate is selected from a compound of monomer unit:

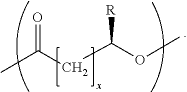

The x is selected from the group consisting of 1 and 2. The R group is selected from the group consisting of hydrogen and alkyl.

In a further embodiment, the ultrafine polyhydroxybutyrate has a maximum particle size below 125 microns.

In a further embodiment, the ultrafine polyhydroxybutyrate has a mean particle size ranging from 0.1 microns to 44 microns.

In a further embodiment, the ultrafine polyhydroxybutyrate has a mean particle size of 5 microns to 20 microns.

In a further embodiment, the ultrafine polyhydroxybutyrate is produced by mechanical milling of coarser powders or pellets.

In a further embodiment, the ultrafine polyhydroxybutyrate is produced by air milling of coarser powders or pellets.

In a further embodiment, the ultrafine polyhydroxybutyrate is produced by wet high shear milling of coarser powders or pellets.

In a further embodiment, the ultrafine polyhydroxybutyrate is produced from a mixture of ultrafine and coarse polyhydroxybutyrate powder by a particle size reduction process.

In a further embodiment, the ultrafine polyhydroxybutyrate is produced directly from a bio-fermentation process, purified, and dried.

In a further embodiment, the ultrafine polyhydroxybutyrate is produced by mechanical or air milling to deagglomerate primary particles and further reduce particle size.

In a further embodiment, the ultrafine polyhydroxybutyrate is produced by wet high shear milling of coarser powders or pellets.

In a further embodiment, the powder further comprises a range of 1 to 50 percent by weight of said ultrafine polyhydroxybutyrate; and a range of 50 to 99 percent by weight of at least one cosmetic powder base components selected from a group consisting of pigments, waxes, solvents, resins, fillers, binders, and mixtures thereof.

In a further embodiment, the powder is loose or compressed and further comprises a range of 5.00 to 10.00 percent by weight of said ultrafine polyhydroxybutyrate; a range of 30.00 to 50.00 percent by weight of talc; a range of 2.00 to 5.00 percent by weight of iron oxide; and 35.00 to 63.00 percent by weight of at least one additive.

The powder for use in cosmetic and personal care products of one embodiment, according to the present teaching, includes ultrafine polyhydroxybutyrate-co-hydroxyvalerate. The ultrafine polyhydroxybutyrate-co-hydroxyvalerate is selected from a compound of monomer unit:

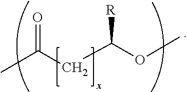

The x is selected from the group consisting of 1, 2, and 3. The R group is selected from the group consisting of hydrogen and alkyl.

In a further embodiment, the ultrafine polyhydroxybutyrate-co-hydroxyvalerate has a maximum particle size below 125 microns.

In a further embodiment, the ultrafine polyhydroxybutyrate-co-hydroxyvalerate has a mean particle size ranging from 0.1 microns to 44 microns.

In a further embodiment, the ultrafine polyhydroxybutyrate-co-hydroxyvalerate has a mean particle size of 5 microns to 20 microns.

In a further embodiment, the ultrafine polyhydroxybutyrate-co-hydroxyvalerate is produced by mechanical milling of coarser powders and pellets.

In a further embodiment, the ultrafine polyhydroxybutyrate-co-hydroxyvalerate is produced by air milling of coarser powders or pellets.

In a further embodiment, the ultrafine polyhydroxybutyrate-co-hydroxyvalerate is produced by wet high shear milling of coarser powders or pellets.

In a further embodiment, the ultrafine polyhydroxybutyrate-co-hydroxyvalerate is produced from a mixture of ultrafine and coarse polyhydroxybutyrate-co-hydroxyvalerate powder by a particle size reduction process.

In a further embodiment, the ultrafine polyhydroxybutyrate-co-hydroxyvalerate is produced directly from a bio-fermentation process, purified, and dried.

In a further embodiment, the ultrafine polyhydroxybutyrate-co-hydroxyvalerate is produced by mechanical or air milling to deagglomerate primary particles and further reduce particle size.

In a further embodiment, the ultrafine polyhydroxybutyrate-co-hydroxyvalerate is produced by wet high shear milling of coarser powders or pellets.

In a further embodiment, the powder comprises a range of 1 to 50 percent by weight of the ultrafine polyhydroxybutyrate-co-hydroxyvalerate; and a range 50 to 99 percent by weight of at least one cosmetic powder base components selected from a group consisting of pigments, waxes, solvents, resins, fillers, binders, and mixtures thereof.

These and other objects of the present invention will become more readily apparent from consideration of the following summary and detailed description. Further areas of applicability of the present invention will become apparent from the detailed description provided hereinafter. It should be understood that the detailed description, while indicating the preferred embodiment of the invention, are intended for purposes of illustration only and are not intended to limit the scope of the invention.

Other embodiments of the system and method are described in detail below and are part of the present teachings.

For a better understanding of the present embodiments, together with other and further aspects thereof, reference is made in the accompanying detailed description, and its scope will be pointed out in the appended claims.

### DETAILED DESCRIPTION

In compliance with the statute, the present teachings have been described in language more or less specific as to structural and methodical features. It is to be understood, however, that the present teaching are not limited to the specific features shown and described, since the systems and methods herein disclosed comprise preferred forms of putting the present teachings into effect.

For purposes of explanation and not limitation, specific details are set forth such as particular architectures, interfaces, techniques, etc. in order to provide a thorough understanding. In other instances, detailed descriptions of well-known devices, circuits, and methods are omitted so as not to obscure the descriptions with unnecessary detail.

The present invention involves the development of novel biodegradable polymer powder in ultrafine form suitable for use in topical applications such as use cosmetic and personal care compositions. The novel biodegradable polymer powder compositions and polymer scrub compositions of the present invention afford good cosmetic properties while being made from annual renewable resources, fully degrade into environmentally friendly constituents and have comparable manufacturing costs as compared to synthetically derived products.

Polyhydroxyalkanoates or PHAs are polyesters produced in nature by numerous microorganisms, including through bacterial fermentation of sugar or lipids. These plastics are biodegradable and are used in the production of bioplastics. Two such bioplastics are PHB (polyhydroxybutyrate homopolymers) and PHBV (polyhydroxybutyrate-co-hydroxyvalerate copolymers), both of which have surprisingly been found to have extremely beneficial properties in the field of cosmetics. They can be either thermoplastic or elastomeric materials, with melting points ranging from 40 to 180° C.

The PHA for use in accordance with the invention are based on hydroxycarboxylic acids as monomer building blocks, which have been polymerized to form polyesters. These monomers or hydroxycarboxylic acids are, in particular, 2-hydroxycarboxylic acids to 6-hydroxycarboxylic acids. This means, for example, that the hydroxyl group is located preferably at least in β-position relative to the carboxyl group of the monomer. The hydroxycarboxylic acids, moreover, may also have different substitutions. Substituents which may be present include for example, but not exclusively, alkyl groups, variously functionalized alkyl, aryl, alkenyl, halogen, cyano, epoxy, ether, acyl, ester, and carboxyl groups, and also combinations of such groups. Preferred monomers present are those in which the PHA comprise at least one kind of the following structures as structural unit or monomer unit:

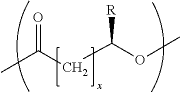

Where: x=1, 2, 3, or 4, more particularly 1 or 2, very preferably 1, and R=H, alkyl, aryl, alkenyl, aralkyl, haloalkyl, halogen, cyano, or radical containing epoxy groups, ether groups, acyl groups, ester groups and/or carboxyl groups, more particularly H or alkyl, and if alkyl then preferably methyl or ethyl, more preferably methyl.

The expression structural unit or monomer unit means that in general a plurality of identical or different monomers, hydroxycarboxylic acids in the context of the present invention, are linked chemically in succession. In the context of the present invention, the chemical linkage comprises ester bonds. A monomer unit, therefore, is a monomer-derived unit in a polymer chain which is present in catenated linkage in the polymer chain with a plurality of further units derived from the same monomer or from different monomers.

PHA used with preference in the context of the present invention comprise one or more blocks each constructed from repeating structural units of the same monomer, the respective monomer being selected from the group consisting of 3-hydroxybutanoic acid, 4-hydroxybutanoic acid, 2-hydroxyvaleric acid, 3-hydroxyvaleric acid, 4-hydroxyvaleric acid, 5-hydroxyvaleric acid, 3-hydroxyhexanoic acid and/or 3-hydroxyoctanoic acid. Employed with particular preference in accordance with the invention are PHA which comprise at least one of the following polymeric and/or copolymeric structures: poly-3-hydroxybutyrate (P-3HB), poly-3-hydroxy-butyrate-co-3-hydroxyvalerate (P-3HB-3HV), poly-3-hydroxybutyrate-co-4-hydroxybutyrate (P-3HB-4HB), and poly-3-hydroxybutyrate-co-3-hydroxyvalerate-co-4-hydroxybutyrate (P-3HB-3HV-4HB). One particularly preferred embodiment of the invention uses PHA which consist of one or more, preferably one, of the aforementioned polymers and copolymers.

The molecular weight of the PHAs for use in the context of the present invention may vary very widely. Thus, the mass-average molecular weights (M_{w}) of the PHA are situated for example in the range from 5,000 to 2,000,000 g/mol (measured by means of gel permeation chromatography (GPC)). The mass average molecular weights (M_{w}) of the PHA are situated preferably in the range from 80,000 to 300,000 g/mol (measured by means of GPC).

### Deriving the Ultrafine Biodegradable Polymer

The inventor has found processes for deriving the ultrafine PHA powder to make them suitable for use in topical applications of the present invention. These approaches are dependent on the specific size and shape of the starting PHA derived from the bio-fermentation process.

The inventor has further found, surprisingly, that the derived ultrafine PHA powder provides highly desirable cosmetic aesthetic properties to products that include loose powders, pressed powders, lotions, creams, and other personal care formulations.

For use in the powder compositions and cosmetic compositions of the present invention, the PHA maximum particle size should be less than 120 mesh or 125 microns. If the PHA starting material is coarser than 120 mesh, the PHA can be processed to the required average particle size according to methods generally known in the art, including, but not limited to, via an air jet mill. A person skilled in the art will be able to determine the necessary parameters for processing the PHA, into the necessary particle size by methods generally known in the art, including by air jet milling, without undue experimentation.

Alternately, the PHA starting material may exist as a complex mixture of ultrafine PHA, including powder and coarser PHA particles. Air classification and/or screen classification processing can be employed, with or without the use of jet mill micronization, to derive the ultrafine PHA powder.

Alternately, the ultrafine PHA powder may be directly derived from the bio-fermentation process, purified as required, dried, and used directly.

Alternately, the ultrafine polyhydroxyalkanoate may be produced by mechanical or air milling to deagglomerate primary particles and further reduce particle size.

In a further embodiment, the ultrafine polyhydroxyalkanoate is produced by wet high shear milling of coarser powders or pellets. High shear wet milling processes could include high shear rotational mixing, 3 roll milling, media milling, or other wet milling processes obvious to those skilled in the art.

### Polymer Powder Compositions of the Invention

Once the PHA is isolated as an ultrafine powder, it can be incorporated into powder compositions suitable for use in cosmetic compositions. The powder compositions of the present invention are preferably ultimately formulated into powder and skincare compositions, but can also be formulated into a wide variety of product types, including, but not limited to, rinses, hand and body lotions, mousses, gels, lotions, tonics, sprays, shampoos, conditioners, facial moisturizers, sunscreens, anti-acne preparations, topical analgesics, mascaras, and the like.

The ultrafine powder compositions of the present invention can be in an amount from 0.5% to 100% by weight ultrafine PHA powder. One or more additional components can be added with the only caveat being that they are cosmetically compatible with the ultrafine PHA powder. These additives can be added in a net amount of from 0.1% to 99.5% and more preferably in the amount of 10% to 50%. Preferred additives include, but are not limited to, waxes, polymers, emulsions, oils, colorants, fragrances, and other binders.

### Cosmetic Compositions of the Invention

The ultrafine powder compositions of the present invention can be formulated into products for topical administration, including into powder cosmetics.

The powder cosmetic compositions of the present invention preferably contains ultrafine PHA powder in an amount of from 1% to 30%, more preferably from 5% to 10% by weight.

Another powder cosmetic compositions of the present invention preferably contains ultrafine PHB powder in an amount of from 1% to 30%, more preferably from 5% to 10% by weight.

Still another powder cosmetic compositions of the present invention preferably contains ultrafine PHBV powder in an amount of from 1% to 30%, more preferably from 5% to 10% by weight.

The powder cosmetic compositions of the present invention preferably contain ultrafine PHA, PHB, or PHBV, powder in an amount of from 1% to 30%, more preferably from 5% to 10% by weight. However, the cosmetic powder compositions may contain a mixture of PHA powders.

The powder compositions according to the invention can have maximum particle size up to 125 microns, preferably with a mean particle size of 5 to 20 microns with a maximum size of 44 microns, and more preferably with a mean particle size of 8 to 12 microns with a maximum particle size of 31.11 microns. The particle size ranges include all specific values and subranges there between. The particle size may be measured with the machine “Microtrac X100 & SRA 150” from Leeds-Northrup as per ASTM D4464 standard.

At a maximum particle size above 125 microns, the powder will not have a good sensation or texture and the user will “feel” the powder particles upon application which will lead to discomfort.

### Other Materials and Additives

Other materials may be present in the compositions of the present invention. In the selection of ingredients in the compositions of the present invention, it is contemplated that materials will be utilized that are compatible, both chemically and physically, with the biodegradable polymers. Such materials include, but are not limited to, cosmetically acceptable diluents or carriers, binders, pigments, pharmacological agents, surfactants, excipients and fillers.

The powder cosmetic compositions herein also comprise one or more cosmetic base powder components selected from pigments, fillers, and binders, and mixtures thereof. It will be appreciated that many of the conventional components of powder cosmetic compositions have more than one functionality and they can therefore be classified under more than one functional types.

Suitable pigments for use herein can be inorganic and/or organic. Also included within the term pigment are materials having a low color or luster such as matte finishing agents, and also light scattering agents. Examples of suitable pigments are iron oxides, acylglutamate iron oxides, ultramarine blue, D&C dyes, carmine, and mixtures thereof.

The total concentration of high luster coloring agents in the powder cosmetic compositions may be from about 0.01% to about 30% by weight, preferably from about 1% to about 15% by weight of the total composition, the exact concentration being dependent to some extent upon the specific mixture of pigments selected to achieve the desired shades. The preferred compositions contain from about 0.1% to about 5% by weight of iron oxides.

Also suitable for use herein especially from the viewpoint of moisturization, skin feel, skin appearance and emulsion compatibility are treated pigments. Pigments can be treated with compounds such as amino acids (e.g., lysine), silicones, lauroyl, collagen, polyethylene, lecithin and ester oils. The more preferred pigments are the silicone (polysiloxane) treated pigments.

The powder compositions can also include at least one matte finishing agent. The function of the matte finishing agent is to hide skin defects and reduce shine. Such cosmetically acceptable inorganic agents, i.e., those included in the CTFA Cosmetic Ingredient Dictionary, Third Ed., include, but are not limited to, silica, hydrated silica, silicone-treated silica beads, mica, talc, polyethylene, calcium silicate, titanium dioxide, bentonite, hectorite, kaolin, chalk, diatomaceous earth, attapugite, zinc oxide and the like may be utilized. Of particular usefulness as a matte finishing agent is low luster pigment such as titanated mica (mica coated with titanium dioxide) coated with barium sulphate. Of the inorganic components useful as a matte finishing agent, low luster pigment, talc, hydrated silica, kaolin, titanium dioxide and mixtures thereof are particularly preferred.

Materials suitable for use herein as light-scattering agents can be generally described as spherical shaped inorganic materials having a particle size of up to about 100 microns, preferably from about 5 to about 50 microns, for example spherical silica particles.

The powder compositions herein can also comprise one or more filler materials. Examples of suitable fillers include, but are not limited to, talc, rice starch and/or bismuth oxychloride, preferably talc. It may also be desirable to include a dry binder in the powder compositions of the invention. Examples of suitable dry binders include, but are not limited to, magnesium stearate, zinc stearate, calcium stearate, lithium stearate, and mixtures thereof. Preferred ranges of the filler materials is from about 5% to about 90% by weight, preferably from about 30% to about 80%, and more preferably from about 60% to about 80% by weight of the total composition.

In addition, the cosmetic compositions of the invention may also comprise any additive usually used in cosmetics and personal care compositions according to the present invention, including, but not limited to, antioxidants, fillers, preserving agents, fragrances, neutralizing agents, thickeners, cosmetic or dermatological active agents including, but not limited to, emollients, moisturizers, vitamins and sunscreens, and mixtures thereof. Preferred ranges of additives is from about 0.5% to about 20% by weight, preferably from about 1% to about 10%, and more preferably from about 2% to about 5% by weight of the total composition.

Examples of possible additives can be found in U.S. Pat. Nos. 7,632,873, 7,670,999, 7,410,636, and 7,351,418 all of which are incorporated by reference in their entirety herein.

Surfactants whether foaming or non-foaming may also be employed in the compositions of the present invention. The foaming surfactants used may be nonionic, anionic, amphoteric or zwitterionic surfactants. A person skilled in the art will be able to choose a surfactant to meet the need of the composition without undue experimentation. Further, examples of foaming surfactants can be found in U.S. Pat. No. 7,655,702 which is specifically incorporated by reference in its entirety herein. Preferred ranges for the surfactants is from about 1% to about 60% by weight, preferably from about 10% to about 50%, and more preferably from about 20% to about 40% by weight of the total composition.

In addition, the cosmetic compositions according to the present invention, can include, but are not limited to, ingredients commonly used in the classical external skin care compositions, skin cleansing compositions, cosmetic compositions and massaging compositions, for example, oily substances, anti-melanogenic agents, sebum secretion inhibitors, blood circulation-facilitating agents, softeners, surfactants, keratin protecting agents, thickeners, antiseptics, pH adjusters, perfume bases, colorants, medicinally-effective agents, solvents, cosmetic or dermatological active agents such as, for example, emollients, moisturizers, vitamins and sunscreens, and mixtures, may be suitably incorporated in addition to the above-described components so far as no detrimental influence is thereby imposed on the effects of the present invention. Preferred ranges for theses additives is from about 1% to about 60% by weight, preferably from about 10% to about 50%, and more preferably from about 20% to about 40% by weight of the total composition.

Other optional ingredients which can be included in the compositions of the invention include preservatives in amounts generally about 1% or less by weight. Suitable preservatives include, but are not limited to methylparaben, propylparaben, imidazolidimyl urea, phenoxyethanol, and mixtures thereof. The compositions may also contain fragrances, sunscreens and chelating agents.

Needless to say, a person skilled in the art will take care to select the optional additional additives and/or the amount thereof such that the advantageous properties of the composition according to the invention are not, or are not substantially, adversely affected by the addition envisaged.

### Manufacture of the Cosmetic Compositions

The composition according to the invention may be manufactured by the known processes generally used in cosmetics or dermatology. For example, the compositions may be prepared by mixing the materials by any conventional means or any means known to one of ordinary skill in the art including, but not limited to, mechanical mixers.

The following examples will more fully illustrate the embodiments of this invention. It will be understood that the following examples are illustrative and not meant to limit the invention in any way. It will also be understood that while the below examples preferably use PHB for its ideal properties, other PHA materials (including, but not limited to PHBV) could also be used. All parts, percentages and proportions referred to herein and in the appended claims are weight unless otherwise illustrated.

### EXAMPLES

### Example 1 Pressed Powder

A pressed powder composition comprising ultrafine polyhydroxybutyrate (PHB) with a mean particle size of 8.0 to 12.0 microns and the ingredients listed below was made according to the following:

TABLE 1

| Product Name | INCI Name | % W/W |
|---|---|---|
| PHASE A | | |
| Talc Micro Ace P-2 | Talc | 40.00 |
| Unipure Yellow LC182 Oxide | Iron Oxide | 1.75 |
| Unipure Brown LC881 Oxide | Iron Oxide | 1.20 |
| Unipure Black LC989 Oxide | Iron Oxide | 0.10 |
| Potassium Sorbate | Potassium Sorbate | 0.20 |
| Tetrasodium EDTA | Tetrasodium EDTA | 0.05 |
| Protachem LL | Lauroyl Lysine | 2.00 |
| Talc Micro Ace P-2 | Talc | 36.40 |
| PHASE B | | |
| Sericite SL-012 | Mica (and) Methicone | 9.00 |
| PHB | Polyhydroxybutyrate | 5.00 |
| PHASE C | | |
| Protachem ISP | Isotearyl Palmitate | 3.55 |
| Vitamin E-Acetate | DL-alpha-tocopheryl acetate | 0.10 |
| Barguard CP | Caprylyl Glycol | 0.75 |
| | Phenoexyethanol, | |
| | Hexylene Glycol | |

The ingredients listed for Phase A were combined in the order listed above. The mixture was passed through a micropulverizer twice such as a Hammermill with a 0.020 or 0.035 herringbone screen or a 4″ Jetmill at 40 g/min (2.4 kg/hr). The powder phase was drawn down to check for streaking. If needed, the powder composition is re-pulverized to ensure uniformity of phase/color. The Phase B materials are then added to the Phase A composition and the combined mixture is blended well with a CBM Mixer (6 quart, tumble speed 35, impeller speed 3,500) for 1 minute until evenly dispersed.

The ultrafine PHB used in the above formulation has a mean particle size of 8.0 to 12.0 microns as measured using a Microtrac device and the ASTM D4464 test method.

The Phase C materials are pre-mixed separately and then added to the Phase AB composition and the resulting composition is blended well with a CBM Mixer (6 quart, tumble speed 35, impeller speed 3,500) for 1 minute until evenly dispersed.

The resulting powder compositions are environmentally friendly and have a good slip resistance and texture. The resulting powder compositions have many and diverse cosmetic applications, including use as foundations, eye shadow, blushes, skincare preparations, mascaras, creams, gels, and lotions.

### Example 2 Loose Face Powder

A loose face powder compositions comprising ultrafine polyhydroxybutyrate (PHB) with a mean particle size of 8.0 to 12.0 microns and the ingredients listed below was made according to the following:

TABLE 2

| Product Name | INCI Name | % W/W |
|---|---|---|
| PHASE A | | |
| Talc Micro Ace P-2 | Talc | 44.04 |
| Unipure Red LC381 Oxide | Iron Oxide | 0.73 |
| Unipure Yellow LC182 Oxide | Iron Oxide | 1.23 |
| Unipure Black LC989 Oxide | Iron Oxide | 0.65 |
| PHASE B | | |
| Sericite SL-012 | Mica (and) Methicone | 5.00 |
| PHB | Polyhydroxybutyrate | 10.00 |
| Talc Micro Ace P-2 | Talc | 30.00 |
| PHASE C | | |
| Dermol 258 | C12-C15 Alkyl Octanoate | 1.75 |
| BHT | Butylated hydroxytoluene | 0.05 |
| Barguard CP | Caprylyl Glycol | 0.75 |
| | Phenoexyethanol, | |
| | Hexylene Glycol | |
| PHASE D | | |
| PresPearl Elegant Gold | Mica (and) Titanium Oxide | 4.00 |
| | (and) Iron Oxide | |
| PresPearl Ruby Red | Mica (and) Iron Oxide | 1.30 |
| PresPearl | Mica (and) Titanium Dioxide | 0.50 |
| | (and) Iron Oxide | |

The ingredients listed for Phase A were combined in the order listed above. The mixture was passed through a micropulverizer twice such as a Hammermill with a 0.020 or 0.035 herringbone screen or a 4″ Jetmill at 40 g/min (2.4 kg/hr). The powder phase was drawn down to check for streaking. If needed, the powder composition is re-pulverized to ensure uniformity of phase/color. The Phase B materials are then added to the Phase A composition and the combined mixture is blended well with a CBM Mixer (6 quart, tumble speed 35, impeller speed 3,500) for 1 minute until evenly dispersed until evenly dispersed.

The ultrafine polyhydroxybutyrate (PHB) used in the above formulation has a mean particle size of 8.0 to 12.0 microns as measured using a Microtrac device and the ASTM D4464 test method.

The Phase C materials are pre-mixed. Slight heat may be required to ensure that the BHT is dissolved in the phase. However, in no event, should the Phase C composition be heated above 40 degrees Celcius. The Phase C composition is then added to the Phase AB composition and the resulting composition is blended well until the materials are evenly dispersed. The Phase D materials are then added to the Phase ABC composition is blended well in a CBM Mixer (6 quart, tumble speed 35, impeller speed 3,500) for 1 minute until evenly dispersed until the materials are evenly dispersed.

The resulting powder compositions are environmentally friendly and have a good slip resistance and texture. The resulting powder compositions have many and diverse cosmetic applications, including use as foundations, eye shadow, blushes, skincare preparations, mascaras, creams, gels, and lotions.

### Example 3 Loose Mineral Powder

A loose mineral powder comprising ultrafine polyhydroxybutyrate-co-hydroxyvalerate (PHBV) with a mean particle size of 8.0 to 12.0 microns and the ingredients listed below was made according to the following:

TABLE 3

| Product Name | INCI Name | % W/W |
|---|---|---|
| PHASE A | | |
| Sericite PHN | Mica | 62.94 |
| Satin B-UVS | Bismuth Oxychloride | 15.00 |
| Unipure Yellow LC182 Oxide | Iron Oxide | 1.73 |
| Unipure Red LC381 Oxide | Iron Oxide | 1.28 |
| Unipure Black LC989 Oxide | Iron Oxide | 0.60 |
| Zinc Stearate | Zinc Stearate | 2.00 |
| PHASE B | | |
| Sericite SL-012 | Mica (and) Methicone | 5.00 |
| PHBV | Polyhydroxybutyrate-co- | 10.00 |
| | hydroxyvalerate | |
| PHASE C | | |
| Ceraphyl 368 | Ethylhexyl Palmitate | 0.50 |
| Dermol 258 | C12-C15 Alkyl Octanoate | 0.35 |
| Barguard CP | Caprylyl Glycol | 0.75 |
| | Phenoexyethanol, | |
| | Hexylene Glycol | |

The ingredients listed for Phase A were combined in the order listed above. The mixture was passed through a micropulverizer twice such as a Hammermill with a 0.020 or 0.035 herringbone screen or a 4″ Jetmill at 40 g/min (2.4 kg/hr). The powder phase was drawn down to check for streaking. If needed, the powder composition is re-pulverized to ensure uniformity of phase/color. The Phase B materials are then added to the Phase A composition and the combined mixture is blended well with a CBM Mixer (6 quart, tumble speed 35, impeller speed 3,500) for 1 minute until evenly dispersed until evenly dispersed.

The ultrafine polyhydroxybutyrate-co-hydroxyvalerate (PHBV) used in the above formulation has a mean particle size 8.0 to 12.0 microns as measured using a Microtrac device and the ASTM D4464 test method.

The Phase C materials are pre-mixed separately and then added to the Phase AB composition and the resulting composition is blended well with a CBM Mixer (6 quart, tumble speed 35, impeller speed 3,500) for 1 minute until evenly dispersed.

The resulting powder compositions are environmentally friendly and have a good slip resistance and texture. The resulting powder compositions have many and diverse cosmetic applications, including use as foundations, eye shadow, blushes, skincare preparations, mascaras, creams, gels, and lotions.

### Example 4 PHB Body Cream/Lotion (Warm Process)

Body cream and body lotion compositions comprising ultrafine polyhydroxybutyrate (PHB) with a mean particle size of 8.0 to 12.0 microns and the ingredients listed below was made according to the following:

TABLE 4

| Product Name | INCI Name | % W/W |
|---|---|---|
| PHASE A | | |
| Xantham Gum | Xantham Gum | 0.50 |
| Vegetable Glycerine | Gylcerine | 5.00 |
| DI Water | Water | 70.80 |
| PHASE B | | |
| Coconut Oil | Cocus Nucifera (Coconut) Oil | 9.00 |
| Procol CS-20-D | Cetearyl Alcohol & Ceteareth-20 | 4.50 |
| Protachem GMS-450 | Glyceryl Steareth | 4.20 |
| PHASE C | | |
| Biosoft 915 (PHB Powder) | Polyhydroxybutyrate | 5.00 |
| PHASE D | | |
| Barguard CP | Caprylyl Glycol Phenoexyethanol, | 0.50 |
| | Hexylene Glycol | |
| Vitamin E Acetate | Vitamin E Acetate | 0.50 |

Weigh Phase A ingredients: DI water, Xantham Gum, and Glycerine into a separate beaker.

Weigh Phase B ingredients: Coconut Oil, Procol CS-20-D, and Protachem GMS-450 into a separate beaker.

Add Phase A into Phase B and use a homogenizer to mix on high speed. Mix the product for 10-20 minutes until it is uniform and homogenous.

Add Phase C (Biosoft 915=PHB) into the batch. Mix the product on high for 15-20 minutes until its uniform and homogenous.

Add Phase D (Barguard CP and Vitamin E Acetate) into the batch and mix for 5-10 minutes. If batch gets thick increased the mixing speed appropriately to keep the batch moving uniformly. Make sure to scrape sides and incorporate in the mix.

### Example 5 PHB Body Cream/Lotion (Cold Process)

Body cream and body lotion compositions comprising ultrafine polyhydroxybutyrate (PHB) with a mean particle size of 8.0 to 12.0 microns and the ingredients listed below was made according to the following:

TABLE 5

| Product Name | INCI Name | % W/W |
|---|---|---|
| PHASE A | | |
| Coconut Oil | Cocus Nucifera (Coconut) Oil | 10.00 |
| Sepinov EMT 10 | Hydroxyethyl acrylate/Sodium | 5.00 |
| | Acryloydimethyl Taurate | |
| | Copolymer | |
| PHASE B | | |
| DI Water | Water | 76.50 |
| Vegetable Based | Glycerine | 2.50 |
| Glycerine | | |
| Vitamin E Acetate | Vitamin E Acetate | 0.50 |
| PHASE C | | |
| Biosoft 915 (PHB Powder) | Polyhydroxybutyrate | 5.00 |
| PHASE D | | |
| Barguard CP | Caprylyl Glycol Phenoexyethanol, | 0.50 |
| | Hexylene Glycol | |

Weigh Phase A ingredients: Coconut Oil and Sepinov EMT 10 into a separate beaker.

Weigh Phase B ingredients: DI water, Glycerine, and Vitamin E Acetate into a separate beaker.

Add Phase A into Phase B and use a homogenizer to mix on high speed. Mix the product for 10-20 minutes until it is uniform and homogenous.

Add Phase C (Biosoft 915=PHB) into the batch. Mix the product on high speed for 15-20 minutes until it is uniform and homogenous.

Add Phase D (Barguard CP) into the batch and mix for 5-10 minutes. If batch gets thick increased the mixing speed appropriately to keep the batch moving uniformly. Make sure to scrape sides and incorporate in the mix.

### Example 6 Loose Face Powder

A loose face powder compositions comprising ultrafine polyhydroxybutyrate (PHB) with a mean particle size of 8.0 to 12.0 microns and the ingredients listed below was made according to the following:

TABLE 6

| INCI Name | % W/W |
|---|---|
| PHASE A | |
| Talc | 30.00-50.00 |
| Iron Oxide | 2.00-5.00 |
| PHASE B | |
| Polyhydroxybutyrate | 5.00-10.00 |
| PHASE C | |
| Various Additives | 35.00-63.00 |

The ingredients listed for Phase A were combined in the order listed above. The mixture was passed through a micropulverizer twice such as a Hammermill with a 0.020 or 0.035 herringbone screen or a 4″ Jetmill at 40 g/min (2.4 kg/hr). The powder phase was drawn down to check for streaking. If needed, the powder composition is re-pulverized to ensure uniformity of phase/color. The Phase B materials are then added to the Phase A composition and the combined mixture is blended well with a CBM Mixer (6 quart, tumble speed 35, impeller speed 3,500) for 1 minute until evenly dispersed until evenly dispersed.

The ultrafine polyhydroxybutyrate (PHB) used in the above formulation has a mean particle size of 8.0 to 12.0 microns as measured using a Microtrac device and the ASTM D4464 test method.

The Phase C materials are pre-mixed. Slight heat may be required to ensure that the BHT is dissolved in the phase. However, in no event, should the Phase C composition be heated above 40 degrees Celcius. The Phase C composition is then added to the Phase AB composition and the resulting composition is blended well until the materials are evenly dispersed. The Phase ABC composition is blended well in a CBM Mixer (6 quart, tumble speed 35, impeller speed 3,500) for 1 minute until evenly dispersed until the materials are evenly dispersed.

The resulting powder compositions are environmentally friendly and have a good slip resistance and texture. The resulting powder compositions have many and diverse cosmetic applications, including use as foundations, eye shadow, blushes, skincare preparations, mascaras, creams, gels, and lotions.

An advantage of the compositions of the present invention is that the compositions are composed of natural polymers as opposed to synthetic polymers. This is particularly desirable since the cosmetic compositions that can be created with the compositions of the present invention are for use on the human body. The compositions of the present invention and the cosmetic compositions created therefrom are ideally suited for use at home as well as at a salon or a spa.

The subsequent use of the powder and powder compositions of the invention depends on the desired aim and on the active agents which may be present. It can be used in particular to care for, cleanse, remove make-up from, make-up and/or treat the human skin, scalp and/or mucous membranes.

Thus, another subject of the present invention is a cosmetic process to care for, cleanse, remove make-up from, make-up and/or treat the skin, mucous membranes and/or the scalp, comprising the application of a powder as defined above to the skin, mucous membranes, and/or the scalp.

Another subject of the invention is the use of the powder as defined above for the preparation of a composition intended to care for, cleanse, remove make-up from, make-up and/or treat the skin, the scalp and/or mucous membranes.

It will therefore be readily understood by those persons skilled in the art that the present invention is susceptible of broad utility and application. Many embodiments and adaptations of the present invention other than those herein described, as well as many variations, modifications, and equivalent arrangements, will be apparent from or reasonably suggested by the present invention and the foregoing description thereof, without departing from the substance or scope of the present invention. Accordingly, while the present invention has been described herein in detail in relation to its preferred embodiment, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications, and equivalent arrangements.

While the present teaching have been described above in terms of specific embodiments, it is to be understood that they are not limited to these disclosed embodiments. Many modifications and other embodiments will come to mind to those skilled in the art to which this pertains, and which are intended to be and are covered by both this disclosure and the appended claims. It is intended that the scope of the present teachings should be determined by proper interpretation and construction of the appended claims and their legal equivalents, as understood by those of skill in the art relying upon the disclosure in this specification.

1. A powder for use in cosmetic and personal care products comprising:
ultrafine polyhydroxyalkanoate;
wherein said ultrafine polyhydroxyalkanoate is selected from a compound of monomer unit: 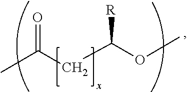
and
wherein x is selected from the group consisting of 1, 2, 3, and 4;
wherein the R group is selected from the group consisting of hydrogen, alkyl, aryl, alkenyl, aralkyl, haloalkyl, halogen, cyano, radical containing epoxy, ether, acyl, ester, and carboxyl;
wherein said powder is compressed.

2. The powder of claim 1, wherein said ultrafine polyhydroxyalkanoate comprise at least one block constructed from monomers, wherein said monomers are selected from the group consisting of 3-hydroxybutanoic acid, 4-hydroxybutanoic acid, 2-hydroxyvaleric acid, 3-hydroxyvaleric acid, 4-hydroxyvaleric acid, 5-hydroxyvaleric acid, 3-hydroxyhexanoic acid, and 3-hydroxyoctanoic acid.

3. The powder of claim 1, wherein said ultrafine polyhydroxyalkanoate is a polymer selected from the group consisting of poly-3-hydroxybutyrate, poly-3-hydroxybutyrate-co-3-hydroxyvalerate, poly-3-hydroxybutyrate-co-4-hydroxybutyrate, and poly-3-hydroxybutyrate-co-3-hydroxyvalerate-co-4-hydroxybutyrate.

4. The powder of claim 1, wherein said ultrafine polyhydroxyalkanoate has a maximum particle size below 125 microns.

5. The powder of claim 1, wherein said ultrafine polyhydroxyalkanoate has a mean particle size ranging from 0.1 microns to 44 microns.

6. The powder of claim 1, wherein said ultrafine polyhydroxyalkanoate has a mean particle size of 5 microns to 20 microns.

7. The powder of claim 1, wherein said ultrafine polyhydroxyalkanoate is produced by mechanical milling of coarser powders or pellets.

8. The powder of claim 1, wherein said ultrafine polyhydroxyalkanoate is produced from a mixture of ultrafine and coarse polyhydroxyalkanoate powder by a particle size reduction process.

9. The powder of claim 1, wherein said ultrafine polyhydroxyalkanoate is produced directly from a bio-fermentation process, purified, and dried.

10. The powder of claim 9, wherein said ultrafine polyhydroxyalkanoate is further produced by a milling process to deagglomerate primary particles and further reduce particle size.

11. The powder of claim 1, wherein said powder further comprises a range of 1 to 50 percent by weight of said ultrafine polyhydroxyalkanoates; and a range of 50 to 99 percent by weight of at least one cosmetic powder base components selected from a group consisting of pigments, waxes, solvents, resins, fillers, binders, and mixtures.

12. A powder for use in cosmetic and personal care products comprising: ultrafine polyhydroxybutyrate, wherein said ultrafine polyhydroxybutyrate is selected from a compound of monomer unit: 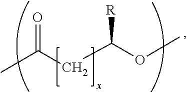
and wherein x is selected from the group consisting of 1 and 2; wherein the R group is selected from the group consisting of hydrogen and alkyl.

13. The powder of claim 12, wherein said ultrafine polyhydroxybutyrate has a maximum particle size below 125 microns.

14. The powder of claim 12, wherein said ultrafine polyhydroxybutyrate has a mean particle size ranging from 0.1 microns to 44 microns.

15. The powder of claim 12, wherein said ultrafine polyhydroxybutyrate has a mean particle size of 5 microns to 20 microns.

16. The powder of claim 12, wherein said ultrafine polyhydroxybutyrate is produced by mechanical milling of coarser powders or pellets.

17. The powder of claim 12, wherein said ultrafine polyhydroxybutyrate is produced from a mixture of ultrafine and coarse polyhydroxybutyrate powder by a particle size reduction process.

18. The powder of claim 12, wherein said ultrafine polyhydroxybutyrate is produced directly from a bio-fermentation process, purified, and dried.

19. The powder of claim 18, wherein said ultrafine polyhydroxyalkanoate is further produced by a milling process to deagglomerate primary particles and further reduce particle size.

20. The powder of claim 12, wherein said powder further comprises a range of 1 to 80 percent by weight of said ultrafine polyhydroxybutyrate; and a range of 50 to 99 percent by weight of at least one cosmetic powder base components selected from a group consisting of pigments, waxes, solvents, resins, fillers, binders, and mixtures.

21. The powder of claim 12, comprising a range of 5.00 to 10.00 percent by weight of said ultrafine polyhydroxybutyrate; a range of 30.00 to 50.00 percent by weight of talc; a range of 2.00 to 5.00 percent by weight of iron oxide; and a range of 35.00 to 63.00 percent by weight of at least one additive.

22. A powder for use in cosmetic and personal care products comprising:
ultrafine polyhydroxybutyrate-co-hydroxyvalerate
wherein said ultrafine polyhydroxybutyrate-co-hydroxyvalerate is selected from a compound of monomer unit: 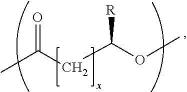
and
wherein x is selected from the group consisting of 1, 2, and 3;
wherein the R group is selected from the group consisting of hydrogen and alkyl;
wherein said powder is compressed.

23. (canceled)

24. The powder of claim 22, wherein said ultrafine polyhydroxybutyrate-co-hydroxyvalerate has a mean particle size ranging from 0.1 micron to 44 microns.

25. The powder of claim 22, wherein said ultrafine polyhydroxybutyrate-co-hydroxyvalerate has a mean particle size of 5 microns to 20 microns.

26. The powder of claim 22, wherein said ultrafine polyhydroxybutyrate-co-hydroxyvalerate is produced by mechanical milling of coarser powders and pellets.

27. The powder of claim 22, wherein said ultrafine polyhydroxybutyrate-co-hydroxyvalerate is produced from a mixture of ultrafine and coarse polyhydroxybutyrate-co-hydroxyvalerate powder by a particle size reduction process.

28. The powder of claim 22, wherein said ultrafine polyhydroxybutyrate-co-hydroxyvalerate is produced directly from a bio-fermentation process, purified, and dried.

29. The powder of claim 28, wherein said ultrafine polyhydroxybutyrate-co-hydroxyvalerate is further produced by a milling process to deagglomerate primary particles and further reduce particle size.

30. The powder of claim 22, wherein said powder comprises a range of 1 to 50 percent by weight of said ultrafine polyhydroxybutyrate-co-hydroxyvalerate; and a range of 50 to 99 percent by weight of at least one cosmetic powder base components selected from a group consisting of pigments, waxes, solvents, resins, fillers, binders, and mixtures.

31. A powder for use in cosmetic and personal care products comprising:
ultrafine polyhydroxybutyrate
wherein said ultrafine polyhydroxybutyrate is selected from a compound of monomer unit: 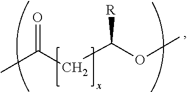
and
wherein x is selected from the group consisting of 1 and 2;
wherein the R group is selected from the group consisting of hydrogen and alkyl;
wherein said powder is loose;
wherein said loose powder is combined with at least oil and water to form an emulsified lotion for topical application.
